(19) United States
(12) Patent Application Publication
(10) Pub. No.: US 2005/0106113 A1
(43) Pub. Date: May 19, 2005

(54) Composition for the treatment of dental and periodontal diseases

(76) Inventors: Harald Greve, Dusseldorf (DE); Rainer Greve, Bad Segeberg (DE)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/984,631

(22) Filed: Nov. 8, 2004

(30) Foreign Application Priority Data
Nov. 17, 2003 (DE) DE10353743.0
Dec. 6, 2003 (DE) DE10357110.8

(51) Int. Cl. A61K 7/16; A61K 7/26; A61K 35/78

(52) U.S. Cl. 424/58; 424/725

(57) ABSTRACT

The invention relates to the use of vervain and/or ribwort, either alone or else as a mixture of the two herbs or of their respective constituents or extracts, for the prophylactic or curative topical treatment of dental and periodontal diseases, such as, for example, dental decay, gingivitis, periodontosis and plaque, and also to a corresponding pharmaceutical composition which contains vervain and/or ribwort or their respective constituents or extracts.

### CROSS-REFERENCE TO RELATED APPLICATION

The present application claims the benefit of German Patent Application Serial No. DE 10353743.0 filed Nov. 17, 2003 and German Patent Application Serial No. DE 10357110.8 filed Dec. 6, 2003, both of which are hereby incorporated by reference in their entirety.

The present invention relates to the use of vervain or ribwort or their plant parts and/or extracts, or else a mixture of the two plants or their plant parts and/or their extracts, for the preparation of suitable products which are intended for exerting a positive effect on the oral flora of humans, in particular for the prophylactic and/or curative topical treatment of dental and periodontal diseases such as dental decay, periodontosis, gingivitis, plaque (dental deposit) and the associated consequences in particular halitosis.

In particular, the present invention relates to the use of vervain and/or ribwort or their extracts or constituents and of a pharmaceutical composition comprising them for the prophylactic and/or curative topical treatment of dental and periodontal diseases such as dental decay, periodontosis, gingivitis, plaque (dental deposit) and associated symptoms or phenomena such as halitosis.

An adverse shift in the oral flora of humans can have very unpleasant consequences, in particular bad breath, up to dental and periodontal diseases such as plaque (dental deposit), dental decay, periodontosis and gingivitis, the multiplication of the following microorganisms being particularly important in this context: Streptococcus mutans, Streptococcus sobrinus, Streptococcus sanguinus, Streptococcus salivarius, Porphyromonas gingivalis, Fusobacterium nucleatum and Actinomyces viscosus.

To date, chemico-synthetic antiseptics such as, for example, hexetidin, dequalinium chloride, chlorhexidine and essential oils have been employed for combating halitosis, plaque, decay, gingivitis and periodontosis, but they are not particularly successful.

Other substances which are employed as alternatives are certain inorganic compounds, such as, for example, aluminium chloride, amine fluoride or tin(II) fluoride; all those are somewhat or moderately successful. However, prolonged use of these substances can lead to staining of the dental enamel and swallowing these substances, for example during gargling, may lead to undesired side effects.

Highly effective, safe, plant-based compositions with a broad range of applications are as yet not available for the effective control of dental and periodontal diseases such as dental decay, periodontosis, gingivitis, plaque (dental deposit) and associated symptoms and phenomena, such as halitosis.

Object of the present invention is thus the provision of a well tolerated composition with a broad range of applications for the prophylaxis or effective therapy of dental and periodontal diseases, in particular dental decay, periodontosis, gingivitis, plaque (dental deposit) and associated symptoms and phenomena, such as halitosis, which composition avoids the above-described problems of the prior art, at least to a substantial extent.

The applicant company has now found that the above object can be achieved entirely surprisingly by using vervain and/or ribwort in suitable preparations.

In a first aspect of the invention, the present invention thus relates to a pharmaceutical composition for the prophylactic and/or curative topical treatment of dental and periodontal diseases (such as dental decay, periodontosis, gingivitis, plaque, or dental deposit, and associated symptoms and phenomena, such as halitosis), which composition comprises, in addition to at least one pharmaceutical excipient, vervain and/or ribwort and/or their constituents and/or extracts in each case in pharmaceutically active amounts.

In a second aspect of the invention, the present invention thus relates to the use of vervain and/or ribwort (in each case alone or in combination) or their respective constituents and/or extracts for the prophylactic and/or curative topical treatment of dental and periodontal diseases such as dental decay, periodontosis, gingivitis, plaque (dental deposit) and associated symptoms and phenomena, such as halitosis, or to the preparation of a pharmaceutical composition for the prophylactic and/or curative topical treatment of dental and periodontal diseases such as dental decay, periodontosis, gingivitis, plaque (dental deposit) and associated symptoms and phenomena, such as halitosis.

Vervain and ribwort are plants which are indigenous in, inter alia, Europe and which have been employed for some time in the form of their herbaceous parts for other indications. All parts of the two plants, that is roots, shoots, leaves and fruits, can be employed for the use in accordance with the invention; however, all of the aerial part (herbaceous part) is preferred. The two plants are commercially available and as such readily obtainable.

Vervain (verbena herb, sometimes also denoted synonymously as “ironweed”), with its typical representative Verbena officinalis, Verbenaceae, is not used widely as a medicinal herb and is traditionally used for example as an astringent and as a bitterant; in addition, it is said to have diuretic, milk-promoting and uterus-contracting effects. Ethanolic extracts have antibacterial and antiviral effects. The plant parts of vervain which are used for pharmaceutical purposes are, in particular, the leaves, which are collected at the time of flowering, and the upper stalk parts. Constituents of vervain are, in particular, verbenalin and other iridoid glycosides, verbascoside and other phenylethanoid glycosides, luteolin-7-O-diglucuronide and other flavonoids, including methoxylated flavones, furthermore stachyose, β-sitosterol and triterpenes. Weakly parasympathomimetic, antiphlogistic and analgesic effects have been identified for verbenalin and for other iridoids.

Ribwort, or its herbaceous part, Plantago lanceolata (angustifolia) herb, Plantaginaceae, is employed in traditional medicine for the secretolysis in catarrhs of the upper respiratory tract. Particularly known is its antitussive action with bronchospasmolitic and expectorant effects. The expressed juice of the fresh herb is also said to be suitable for external use to promote wound healing and against insect bites, owing to its antibacterial and anti-inflammatory action. The plant parts which are used in particular for pharmaceutical purposes are the plantain herb, Plantago lanceolata herb, which are the aerial parts which are harvested at the time of flowering and dried rapidly. Constituents of plantain are, in particular, aucubin, catalpol and other iridoid glycosides, acteosid and further phenylethanoids, phenol carboxylic acids such as, for example, caffeic acid, ferulic acid, furthermore flavonoids, tannins, mucus polysaccharides, silica, essential oils and vitamin C.

Heretofore, however none of the two plants has been considered for the treatment of dental and periodontal diseases (for example dental decay, gingivitis, periodontosis and plaque, or dental deposit, and associated symptoms or phenomena, such as halitosis). It was therefore entirely surprising, and has been found for the first time by the applicant company, that vervain and/or ribwort, either alone or in combination, or their respective constituents or extracts are outstandingly suitable for the prophylactic and/or curative topical therapy of dental and periodontal diseases (for example dental decay, gingivitis, periodontosis and plaque, or dental deposit, and associated symptoms or phenomena, such as halitosis).

The pharmaceutical composition according to the invention preferably contains vervain and/or ribwort or their constituents and/or extracts as the sole active ingredient(s), for example in the form of comminuted, in particular powdered, plant constituents and/or in the form of their extracted constituents and/or in the form of extracts.

However, it is not ruled out that the pharmaceutical composition according to the invention additionally contains further active ingredient(s) and/or constituents. For example, the pharmaceutical composition, or preparation, according to the invention may also contain other active ingredients or constituents, or mixtures of further active ingredients or constituents, which may be of plant origin or else of nonplant origin and which do not adversely affect the effect of vervain and/or ribwort or indeed enhance their effect. In addition, substances or substance mixtures which improve the flavour and/or odour and the technological application of the pharmaceutical composition according to the invention may additionally be added.

A combination of the two herbs (i.e. vervain and ribwort) or of the constituents or extracts of the two herbs has proved to be particularly effective.

For example, the pharmaceutical composition according to the invention may contain vervain and/or ribwort in the form of their respective extracts, in particular in the form of liquid or dry extracts, the preferred herb/extract ratio being 50:1 to 1:1, especially preferably 10:1 to 1:0.2. For example, the pharmaceutical composition of the present invention contain vervain and/or ribwort in the form of liquid extracts (for example in the form of alcoholic or alcoholic-aqueous extracts, preferably in the form of ethanolic or ethanolic-aqueous extracts), or else in the form of solid extracts.

In this context, the pharmaceutical composition of the present invention can be liquid, gel-like, pasty, ointment-like or solid, depending on the intended application. For example, the pharmaceutical composition according to the invention can be present for example in the form of lozenges/chewable tablets or (chewable) pastilles or else in the form of a solution or dispersion which can be used for topical application in the oropharyngeal cavity, in particular a mouthwash and/or gargle solution, or in the form of ointments, creams, gels or pastes which can be used for topical application in the oropharyngeal cavity, in particular toothpastes.

The amounts of vervain and/or ribwort or their constituents or extracts in the pharmaceutical composition according to the invention can vary within wide limits. In general, the pharmaceutical composition according to the invention may contain vervain and/or ribwort or their constituents and/or extracts, in amounts of from at least 0.1% by weight, in particular at least 1% by weight, preferably at least 5% by weight, particularly preferably at least 10% by weight, and in amounts of up to 50% by weight, in particular of up to 75% by weight, preferably of up to 90% by weight, especially preferably of up to 95% by weight or even more, in each case based on the amount of the original plant material and based on the pharmaceutical composition. If appropriate, however, it may be necessary to deviate from the above-mentioned amounts if required by the therapeutical circumstances.

In addition to the actual, vervain- and/or ribwort-based active ingredients, the pharmaceutical composition of the present invention may also contain customary pharmaceutical additives and/or adjuvants, in particular fillers, extenders, binders, wetting agents, stabilizers, colorants, buffers, aroma chemicals, flavourings and/or preservatives.

The two plants, or the parts of the two plants (in each case either alone or in combination with each other)—also in comminuted form, down to a powder—can be employed as such in the use according to the invention. The use in the form of extracts or constituents of the two plants is, however, preferred.

As described above, the extracts may be solid or dry, or else liquid. Liquid extracts may, in addition to the plant-based active ingredients, contain liquid extractants with which the skilled worker is familiar. Preferred are water, alcohols, in particular potable alcohol (ethanol) or ethanol-water mixtures are preferred. In addition to the plant-based active ingredients or constituents, solid or dry, extracts may contain in particular inert solids such as, for example, silicon dioxide or xylitol.

The extracts have a preferred herb/extract ratio of from 50:1 to 1:1, especially preferably from 10:1 to 1:0.2. The extracts can be prepared in a manner known per se to the skilled worker, for example in such a way that 0.2 to 100 parts, preferably 1 part, of liquid extract, or 0.1 part of dry extract, are obtained from, for example, 1 part of dried or fresh plant material (herbal matter) with the aid of suitable liquid extractants, preferably ethanol/water. The amount of extract required for achieving the desired herb/extract ratio can be standardized; in the case of liquid extracts by direct extraction followed by concentration, if required; in the case of dry extracts by evaporating liquid extracts to dryness and subsequent mixing with inert solids. The skilled worker is familiar with this procedure per se; no further explanations are therefore required.

Preparations containing vervain, ribwort or their extracts can be liquid, gel-like or pasty and, in addition to the plant constituents, or extractive substances, the acceptable diluents which are compatible with vervain and/or ribwort which are conventionally used in production technology and other adjuvants.

A preparation which has proved particularly suitable for the prophylaxis and treatment of plaque formation, gingivitis, dental decay and periodontosis is the principle of a gargle rinse containing approximately 2.5% of an extract which contains approximately 60% ethanol (% by volume). Approximately 20 drops of this preparation per half a glass of water (approximately 50 ml) suffice for achieving adequate inhibition of microorganisms.

To achieve a sufficient effect in the semi-solid forms, such as, for example, toothpaste or oral gels, such forms can likewise contain approximately 2.5% of an extract which contains approximately 80% ethanol (% by volume).

In the case of lozenges, chewable tablets and (chewable) pastilles it is preferred that such preparations contain approximately 250 mg of dry extract per unit. In the most advantageous case, such a dry extract will have been prepared by concentrating the liquid extract which originally has a herb/extract ratio of approximately 1:1 and an alcohol component of approximately 80% (% by volume).

Further embodiments, modifications, variations and advantages of the present invention are immediately recognizable to the skilled worker on reading the description and are realisable by the skilled worker without departing from the scope of the present invention.

The present invention is illustrated using the examples hereinbelow, which do not, however, in any way restrict the invention.

### USE EXAMPLES

### Example 1

To prepare a liquid extract from vervain with a herb/extract ratio of 1:1, 100 g of commercially available, dried and chopped vervain are extracted by percolation using the method described in DAB [German Pharmacopoeia] 9, page 800, in the monograph “Extrakte” [Extract]. First, the herb is powdered coarsely by grinding, and the powder is then moistened uniformly with 30 g of ethanol (70% by volume) in an open dish, where it is left to stand for two hours for presoaking. After sieving, the percolator specified in DAB 9 is charged with the mixture, and the percolation is carried out by the method described therein, using 70% ethanol (% by volume). The percolation is finished when 85 g of percolate have been collected. The drainage stop-cock is then turned off, the further application of ethanol (70% by volume) is stopped and the herbal residue is subjected to expression. The expression fluid is filtered, concentrated in vacuo on a rotary evaporator and added to the percolate. After the product has been left to stand for several days in a sealed vessel at approximately 5 to 10° C., it is checked for clarity and, if necessary, filtered. This gives a vervain-based liquid extract with a herb/extract ratio of 1:1.

The extract is suitable for the preparation of a mouthwash/gargle concentrate which contains approximately 2.5% of the extract which contains approximately 60% of ethanol (% by volume) and water as the remainder. Using approximately 15 to 20 drops of this preparation per approximately 50 ml suffices for achieving sufficient inhibition of microorganisms and thus on effective prophylaxis or therapeutic treatment of gingivitis, periodontosis, dental decay or plaque when used several times a day.

### Example 2

Example 1 is repeated, except that the vervain is replaced completely by the herbaceous parts of ribwort. The extract, or the mouthwash/gargle concentrate prepared on the basis of this extract, is equally outstandingly suitable for the prophylactic and therapeutic treatment of dental and periodontal diseases, in particular dental decay, gingivitis, periodontosis and plaque.

### Example 3

Example 1 is repeated, except that 50 g of vervain are replaced by the herbal parts of ribwort. An extract which is based on a combination of vervain and ribwort results. The extract, or the mouthwash/gargle concentrate prepared on the basis of this extract, are likewise suitable for the treatment of dental and periodontal diseases.

### Examples 4 to 6

Examples 1 to 3 are modified in such a way that, starting from the aqueous-alcoholic extracts described in Examples 1 to 3, dry extracts are prepared which are subsequently processed in a manner known per se together with the excipients and adjuvants conventionally used for this purpose to give lozenges or chewable tablets (herb/extract ratio in each case 1:1, in each case 250 mg of dry extract per unit, or tablet).

The lozenges or chewable tablets prepared in this manner are likewise suitable for the treatment of the above-mentioned type of dental and periodontal diseases.

### Example 7

Different liquid ethanolic extracts on the basis of vervain (=fluid extracts “V30”, “V50” and “V80”) and on the basis of ribwort (=fluid extracts “P30”, “P50” and “P80”) as well as on the basis of vervain plus ribwort (=fluid extract “V80 plus P80”), each of them with different concentrations, were examined with respect to their effectiveness in relation to the inhibition of the growth of certain pathogenous germs (bacteria) of the oral flora (mouth flora).

| | | |
|---|---|---|
| Sample: | fluid extract V30 | actual ethanol content: 24.3% |
| | fluid extract V50 | actual ethanol content: 41.4% |
| | fluid extract V80 | actual ethanol content: 63.3% |
| | fluid extract P30 | actual ethanol content: 20.2% |
| | fluid extract P50 | actual ethanol content: 38.6% |
| | fluid extract P80 | actual ethanol content: 63.8% |

1:1-mixture V80 plus P80 (vervain plus ribwort)

Several samples according to the present invention were examined with respect to their ability to inhibit the growth of pathogenous germs of the oral flora. In these examinations, the so-called minimal inhibition concentrations (MIC) were determined with respect to six single fluid samples as well as to a 1:1-mixture of the fluid extract of V80 and P80, i. e. the lowest concentrations of these extracts and this mixture, respectively, which inhibit the growth of particular bacteria (germs) were determined. For this purpose, bacteria of the human oral flora which may cause dental and periodontal infections were chosen (see Table 1). The bacterium Streptococcus mutans which is mainly responsible for the development of caries belongs to these bacteria, for example.

The realization of the tests was performed according to standardized DIN-methods 58940 and 58944 (standardized German Industrial Norm [Deutsche Industrie-Norm]). Beginning with the highest concentration (10%) of the different fluid extracts ten concentrations were tested individually.

The results are shown in Tables 2 to 8. The “minimal inhibition concentration” (MIC) is defined as the concentration of the active substance at which macroscopically no growth of the tested bacteria is observed. In general, the determination of the MIC is associated with a limit error up to two dilution levels.

TABLE 1
Selected germs of the oral flora and their pathogenous potential

| germ | pathogenous potential |
|---|---|
| Streptococcus mutans | caries germ (pathogen) |
| Streptococcus sobrinus | caries germ (pathogen) |
| Streptococcus sanguis | indirect, film on the teeth (plaque) |
| Streptococcus salivarius | rarely pathogen (caries) |
| Porphyromonas gingivalis | paradontosis |
| Fusobacteria nucleatum | dentogenous suppurations, paradontosis |
| Actinomyces viscosus | caries, paradontosis |

Under the specified experimental conditions an inhibitory effect with respect to the growth of bacteria was observed. In comparison with the fluid extracts V30, V50 and V80 and P30, P50 and P80, respectively, the lowest MICs for nearly all bacteria were observed for the fluid abstracts V80 and P80, respectively. That means that the fluid extracts V80 and P80 were the most effective extracts with respect to the inhibition of the growth of tested bacteria under the specified experimental conditions.

The lowest effective concentrations with respect to the tested bacteria were found to be in the following ranges:
V80: 0.63-2.5%; for the caries pathogen Streptococcus mutans at 2.5%.
P80: 0.63-5%; for the caries pathogen Streptococcus mutans at 5%.

The 1:1-mixture of the fluid extract V80 plus P80 (fluid extract on the basis of vervain and ribwort) was even more effective than the individual extracts (see Table 8). Here, the lowest efficient concentrations for the tested bacteria were in a significant lower range (0.16-0.63% of the 1:1-mixture). With respect to Streptococcus mutans, the observed minimal inhibition concentration was at 0.31 %. Thus, compared with the individual fluid extracts, a 10-fold lower concentration led still to an inhibition of the growth of this bacterium under the chosen experimental conditions.

The higher effectiveness of the 1:1-mixture of V80 plus P80 shows a synergistic effect, that means the fluid extracts of the two herbs in combination (vervain and ribwort) enhance each other in their effectiveness.

TABLE 2
Experimental results of MIC-determination of the sample “fluid extract V30”

| | final concentrationin % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | ++ | ++ | ++ | ++ | ++ | ++ | ++ | ++ | 5 |
| viscosus | | | | | | | | | | | |
| Fusobacterium | −− | ++ | ++ | ++ | ++ | ++ | ++ | ++ | ++ | ++ | 10 |
| nucleatum | | | | | | | | | | | |
| Porphyromonas | −− | ++ | ++ | ++ | ++ | ++ | ++ | ++ | ++ | ++ | 10 |
| gingivalis | | | | | | | | | | | |
| Streptococcus | −− | ++ | ++ | ++ | ++ | ++ | ++ | ++ | ++ | ++ | 10 |
| mutans | | | | | | | | | | | |
| Streptococcus | −− | ++ | ++ | ++ | ++ | ++ | ++ | ++ | ++ | ++ | 10 |
| salivarius | | | | | | | | | | | |
| Streptococcus | −− | ++ | ++ | ++ | ++ | ++ | ++ | ++ | ++ | ++ | 10 |
| sanguinis | | | | | | | | | | | |
| Streptococcus | −− | ++ | ++ | ++ | ++ | ++ | ++ | ++ | ++ | ++ | 10 |
| sobrinus | | | | | | | | | | | |

+ = growth,
− = no growth

TABLE 3
Experimental results of MIC-determination of the sample “fluid extract V50”

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | −− | −− | −− | ++ | ++ | ++ | ++ | ++ | 0.63 |
| viscosus | | | | | | | | | | | |
| Fusobacterium | −− | −− | −− | −− | ++ | ++ | ++ | ++ | ++ | ++ | 1.25 |
| nucleatum | | | | | | | | | | | |
| Porphyromonas | −− | −− | −− | −− | −− | ++ | ++ | ++ | ++ | ++ | 0.63 |
| gingivalis | | | | | | | | | | | |
| Streptococcus | −− | −− | ++ | ++ | ++ | ++ | ++ | ++ | ++ | ++ | 5 |
| mutans | | | | | | | | | | | |
| Streptococcus | −− | −− | −− | ++ | ++ | ++ | ++ | ++ | ++ | ++ | 2.5 |
| salivarius | | | | | | | | | | | |
| Streptococcus | −− | −− | −− | ++ | ++ | ++ | ++ | ++ | ++ | ++ | 2.5 |
| sanguinis | | | | | | | | | | | |
| Streptococcus | −− | −− | −− | ++ | ++ | ++ | ++ | ++ | ++ | ++ | 2.5 |
| sobrinus | | | | | | | | | | | |

+ = growth,
− = no growth

TABLE 4
Experimental results of MIC-determination of the sample “fluid extract V80”

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | −− | −− | +− | ++ | ++ | ++ | ++ | ++ | 1.25 |
| viscosus | | | | | | | | | | | |
| Fusobacterium | −− | −− | −− | −− | −− | ++ | ++ | ++ | ++ | ++ | 0.63 |
| nucleatum | | | | | | | | | | | |
| Porphyromonas | −− | −− | −− | −− | −− | ++ | ++ | ++ | ++ | ++ | 0.63 |
| gingivalis | | | | | | | | | | | |
| Streptococcus | −− | −− | −− | ++ | ++ | ++ | ++ | ++ | ++ | ++ | 2.5 |
| mutans | | | | | | | | | | | |
| Streptococcus | −− | −− | −− | −− | −− | ++ | ++ | ++ | ++ | ++ | 0.63 |
| salivarius | | | | | | | | | | | |
| Streptococcus | −− | −− | −− | −− | +− | ++ | ++ | ++ | ++ | ++ | 1.25 |
| sanguinis | | | | | | | | | | | |
| Streptococcus | −− | −− | −− | −− | −− | ++ | ++ | ++ | ++ | ++ | 0.63 |
| sobrinus | | | | | | | | | | | |

+ = growth,
− = no growth

TABLE 5
Experimental results of MIC-determination of the sample “fluid extract P30”

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | +− | ++ | ++ | ++ | ++ | ++ | ++ | ++ | ++ | 10 |
| viscosus | | | | | | | | | | | |
| Fusobacterium | −+ | ++ | ++ | ++ | ++ | ++ | ++ | ++ | ++ | ++ | >10 |
| nucleatum | | | | | | | | | | | |
| Porphyromonas | −− | −− | ++ | ++ | ++ | ++ | ++ | ++ | ++ | ++ | 5 |
| gingivalis | | | | | | | | | | | |
| Streptococcus | −− | −− | ++ | ++ | ++ | ++ | ++ | ++ | ++ | ++ | 5 |
| mutans | | | | | | | | | | | |
| Streptococcus | −− | −− | ++ | ++ | ++ | ++ | ++ | ++ | ++ | ++ | 5 |
| salivarius | | | | | | | | | | | |
| Streptococcus | −− | −+ | ++ | ++ | ++ | ++ | ++ | ++ | ++ | ++ | 10 |
| sanguinis | | | | | | | | | | | |
| Streptococcus | −− | −− | ++ | ++ | ++ | ++ | ++ | ++ | ++ | ++ | 5 |
| sobrinus | | | | | | | | | | | |

+ = growth,
− = no growth

TABLE 6
Experimental results of MIC-determination of the sample “fluid extract P50”

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | −− | −− | ++ | ++ | ++ | ++ | ++ | ++ | 1.25 |
| viscosus | | | | | | | | | | | |
| Fusobacterium | −− | −− | −− | ++ | ++ | ++ | ++ | ++ | ++ | ++ | 2.5 |
| nucleatum | | | | | | | | | | | |
| Porphyromonas | −− | −− | −− | −− | ++ | ++ | ++ | ++ | ++ | ++ | 1.25 |
| gingivalis | | | | | | | | | | | |
| Streptococcus | −− | −− | ++ | ++ | ++ | ++ | ++ | ++ | ++ | ++ | 5 |
| mutans | | | | | | | | | | | |
| Streptococcus | −− | −− | −− | ++ | ++ | ++ | ++ | ++ | ++ | ++ | 2.5 |
| salivarius | | | | | | | | | | | |
| Streptococcus | −− | −− | −− | ++ | ++ | ++ | ++ | ++ | ++ | ++ | 2.5 |
| sanguinis | | | | | | | | | | | |
| Streptococcus | −− | −− | −− | ++ | ++ | ++ | ++ | ++ | ++ | ++ | 2.5 |
| sobrinus | | | | | | | | | | | |

+ = growth,
− = no growth

TABLE 7
Experimental results of MIC-determination of the sample “fluid extract P80”

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | −− | −− | −− | ++ | ++ | ++ | ++ | ++ | 0.63 |
| viscosus | | | | | | | | | | | |
| Fusobacterium | −− | −− | −− | −− | ++ | ++ | ++ | ++ | ++ | ++ | 1.25 |
| nucleatum | | | | | | | | | | | |
| Porphyromonas | −− | −− | −− | −− | −− | ++ | ++ | ++ | ++ | ++ | 0.63 |
| gingivalis | | | | | | | | | | | |
| Streptococcus | −− | −− | ++ | ++ | ++ | ++ | ++ | ++ | ++ | ++ | 5 |
| mutans | | | | | | | | | | | |
| Streptococcus | −− | −− | −− | −− | +− | ++ | ++ | ++ | ++ | ++ | 1.25 |
| salivarius | | | | | | | | | | | |
| Streptococcus | −− | −− | −− | −− | −− | ++ | ++ | ++ | ++ | ++ | 0.63 |
| sanguinis | | | | | | | | | | | |
| Streptococcus | −− | −− | −− | −− | −− | ++ | ++ | ++ | ++ | ++ | 0.63 |
| sobrinus | | | | | | | | | | | |

+ = growth,
− = no growth

TABLE 8
Experimental results of MIC-determination of the sample “fluid extract V80
plus P80” (1:1-mixture)

| | final concentration in % | | | | | | | | | | MIC |
|---|---|---|---|---|---|---|---|---|---|---|---|
| test germ | 10 | 5 | 2.5 | 1.25 | 0.63 | 0.31 | 0.16 | 0.08 | 0.04 | 0.02 | in % |
| Actinomyces | −− | −− | −− | −− | −− | +− | ++ | ++ | ++ | ++ | 0.63 |
| viscosus | | | | | | | | | | | |
| Fusobacterium | −− | −− | −− | −− | −− | −− | +− | ++ | ++ | ++ | 0.31 |
| nucleatum | | | | | | | | | | | |
| Porphyromonas | −− | −− | −− | −− | −− | −− | −− | ++ | ++ | ++ | 0.16 |
| gingivalis | | | | | | | | | | | |
| Streptococcus | −− | −− | −− | −− | −− | −− | +− | ++ | ++ | ++ | 0.31 |
| mutans | | | | | | | | | | | |
| Streptococcus | −− | −− | −− | −− | −− | −− | ++ | ++ | ++ | ++ | 0.31 |
| salivarius | | | | | | | | | | | |
| Streptococcus | −− | −− | −− | −− | −− | −− | −+ | ++ | ++ | ++ | 0.31 |
| sanguinis | | | | | | | | | | | |
| Streptococcus | −− | −− | −− | −− | −− | +− | ++ | ++ | ++ | ++ | 0.63 |
| sobrinus | | | | | | | | | | | |

+ = growth,
− = no growth

1. A pharmaceutical composition for the prophylactic and curative topical treatment of dental and periodontal diseases, said pharmaceutical composition comprising:
at least one pharmaceutical excipient; and
at least one of vervain, ribwort, extracts of vervain, and extracts of ribwort, in each case in pharmaceutically effective amounts.

2. The pharmaceutical composition according to claim 1, wherein the at least one of vervain, ribwort, extracts of vervain, and extracts of ribwort is the sole active ingredient(s).

3. The pharmaceutical composition according to claim 1, containing vervain or ribwort or both in the form of comminuted or powdered plant components or parts.

4. The pharmaceutical composition according to claim 1, containing at least one of vervain or ribwort or in the form of their extracts.

5. The pharmaceutical composition according to claim 4, containing vervain or ribwort, or both in the form of their respective extracts having an herb/extract ratio of from 50:1 to 1:1.

6. The pharmaceutical composition according to claim 4, containing vervain or ribwort, or both in the form of their respective extracts, having an herb/extract ratio of from 10:1 to 1:0.2.

7. The pharmaceutical composition according to claim 1, wherein the pharmaceutical composition contains at least one of vervain, ribwort, and their extracts, in pharmaceutically effective amounts of from at least 0.1% by weight up to 95% by weight or even more, in each case based on the amount of original plant material and based on the pharmaceutical composition.

8. The pharmaceutical composition according to claim 1, wherein the pharmaceutical composition contains at least one of vervain, ribwort, extracts of vervain and extracts of ribwort, in amounts of from at least 1% by weight up to 90% by weight, in each case based on the amount of original plant material and based on the pharmaceutical composition.

9. The pharmaceutical composition according to claim 1, wherein the pharmaceutical composition is liquid, gel, paste, ointment or solid.

10. The pharmaceutical composition according to claim 1, wherein the pharmaceutical composition exists in the form of lozenges, chewable tablets, chewable pastilles, in the form of a solution, or a dispersion formulated to be used for topical application in the oropharyngeal cavity.

11. The pharmaceutical composition according to claim 1 formulated for the prophylactic and/or curative topical treatment of dental decay, gingivitis, periodontosis, plaque, dental deposit, and associated symptoms including halitosis.

12. The pharmaceutical composition for the prophylactic and curative topical treatment of dental and periodontal diseases, said pharmaceutical composition comprising:
at least one pharmaceutical excipient, and
a combination of vervain and ribwort, or a combination of their extracts in each case in pharmaceutically-effective amounts.

13. Pharmaceutical composition according to claim 12, containing vervain and ribwort or in the form of their extracts.

14. The pharmaceutical composition according to claim 13, containing extracts of vervain and ribwort, wherein the extracts have a herb/extract ratio from 50:1 to 1:1.

15. The pharmaceutical composition according to claim 13, containing extracts of vervain and ribwort, wherein the extracts have a herb/extract ratio from 10:1 to 1:0.2.

16. A method of treating a human suffering from a dental and periodontal disease, said method comprising administering an effective amount of at least one of vervain, ribwort, and their extracts to said human by topical application.

17. A method of treating a human suffering from a dental and periodontal disease, said method comprising administering an effective amount of a combination of vervain and ribwort or of their extracts to said human by topical application.

18. A method of treating a human suffering from a dental and periodontal disease, said method comprising administering an effective amount of the pharmaceutical composition as defined in claim 1 to said human by topical application.

19. A method of treating a human suffering from a dental and periodontal disease, said method comprising administering an effective amount of the pharmaceutical composition as defined in claim 12 to said human by topical application.
